(19) United States
(12) Patent Application Publication
(10) Pub. No.: US 2008/0153785 A1
(43) Pub. Date: Jun. 26, 2008

(54) Pharmaceutical Compositions Containing Biophosphonate for Improving Oral Absorption

(75) Inventors: Hee-Jong Shin, Gyeonggi-do (KR); Min-Hyo Kim, Chungcheongnam-do (KR); Mee-Hwa Choi, Chungcheongnam-do (KR); Jin-Woo Lee, Chungcheongnam-do (KR)

Correspondence Address: THE WEBB LAW FIRM, P.C., 700 KOPPERS BUILDING, 436 SEVENTH AVENUE, PITTSBURGH, PA, 15219, US

(73) Assignee: CHONG KUN DANG PHARMACEUTICAL CORP., Seoul (KR)

(21) Appl. No.: 11/883,033

(22) PCT Filed: Jan. 3, 2006
(86) PCT No.: PCT/KR2006/000013

§ 371 Date: Jul. 25, 2007

(30) Foreign Application Priority Data
Jan. 31, 2005 (KR) 10-2005-0008711

(51) Int. Cl. A61K 31/663; A61P 19/10

(52) U.S. Cl. 514/108; 514/102

(57) ABSTRACT

Provided is a pharmaceutical composition having improved oral absorption of a bisphosphonate drug, comprising at least one active substance selected from bisphosphonate drugs having non-permeability to a lipid biomembrane due to ionization and high water-solubility in vivo and having bioavailability of less than 10%, and at least one selected from biocompatible water-soluble chitosan, as essential ingredients.

### TECHNICAL FIELD

The present invention relates to a pharmaceutical composition of a bisphosphonate drug, containing water-soluble, high-molecular weight chitosan and/or low-molecular weight chitosan and/or a chitosan monomer, which are soluble in water. Therefore, the present invention provides a novel pharmaceutical composition for improving an oral absorption rate of a drug having low bioavailability due to non-permeability to a lipid biomembrane resulting from high polarity thereof.

### BACKGROUND ART

Bisphosphonate drugs, which are currently under development, exhibit non-permeability to lipid biomembranes due to excessively high polarity thereof. In addition, it is known that such bisphosphonate compounds have high affinity for multivalent metal ions such as calcium, and therefore suffer from difficulty of absorption via cell membranes in digestive tracts due to formation of insoluble complexes upon binding between the drugs and metal ions in vivo (Br. J. Cancer, 71, Suppl. 24, 67, 1995). Further, it is reported that the bisphosphonate compounds have anionic properties inside small intestines having a pH ranging from 6 to 8, which leads to difficult absorption in small intestines, and as a result, most drugs have low absorption rate of less than 10% and particularly an absorption rate of alendronate sodium is less than 1% (Clin pharmacol & Therapeutics, 58(3), 288-209 (1995)).

There are reported some of studies for increasing absorption of bisphosphonate drugs, which are conventionally known to have lower bioavailability due to incapability of free diffusion and permeation which are absorption mechanisms of lipid biomembranes of the gastrointestinal (GI) tracts, resulting from high polarity thereof, and also difficulty of absorption via intercellular space.

For example, as prior arts of increasing an oral absorption rate of the drug by adding an absorption enhancer including a fatty acid to an active substance including bisphosphonate, reference may be made to U.S. Patent Laid-Open Publication No. 2003/0091623 and WO 00/50012. In addition, WO 99/18972 discloses a pharmaceutical formulation using medium chain triglyceride as an absorption enhancing agent and WO 01/76577 discloses a composition of bisphosphonate using a zwitterionic phospholipid as the an absorption enhancing agent. All of the above patents design solid preparations using fat-derived materials and therefore suffer from numerous shortcomings. In particular, fatty acid and medium chain triglyceride are of liquid or semi-solid and should be used in a much larger amount than an active substance, thus leading to difficulty in practical commercialization of preparations. In addition, the zwitterionic phospholipid also leads to difficulty in practical commercialization of preparations, due to its low stability and risk of putrefaction.

U.S. Pat. No. 6,309,663 proposes a method for enhancing absorption of a therapeutic agent via use of a combination of a hydrophilic surfactant and a hydrophobic surfactant. This patent has claimed a comprehensive range of chitosan derivatives while covering a very broad range of surfactants, and thus the presence of surfactant itself may be another cause for harmful side effects on gastrointestinal tracts. Whereas, the present composition contains no surfactants and is helpful in alleviation of gastrointestinal disorders. In addition, the cited invention uses chitosan-EDTA and/or chitosan-EDTA-antipain complexes which have properties significantly different from those inherent to chitosan and these complexes are used as an enzyme inhibitor. Therefore, upon considering the above-mentioned points, the present invention is definitely different from U.S. Pat. No. 6,309,663.

WO 02/070438 discloses a method for enhancing oral absorption of a drug via use of a self-developed oral absorption enhancing agent which is different from the water-soluble chitosan used in the present invention. The synthetic absorption enhancing agent used in WO 02/070438 is not a biocompatible substance and may thus exhibit adverse side effects on the gastrointestinal tract. Therefore, it cannot be said that such an absorption enhancing agent is a medical grade excipient commonly used in the art.

Meanwhile, WO 00/61111 claims a composition having a very broad range of excipients as a pharmaceutical formulation for providing an enhanced absorption of bisphosphonate, without demonstration of absorption-improving effects of the drug. Excipients illustrated therein include and use at least one substance selected from all kinds of surfactants; a bile acid; fatty acid and saponification salts thereof; medium chain glyceride; oil; enamine; all kinds of chelating agents; phenothiazine; fatty acid derivatives of carnitine or peptide; substances selected from the group consisting of azone, concanavalin A, phosphate and derivatives thereof and diphosphate and derivatives thereof; products from Maillard reactions; all kinds of polymers including copolymers and biodegradable polymers; and chitosan and chitosan derivatives. However, there is no example providing evidence of enhanced absorption effects in the form of concrete data, while enumerating the composition only. Further, chitosan correlating to the present invention is not mentioned in any examples of composition. Even though the gastrointestinal environment is acidic, there is high possibility that chitosan may not be dissolved since the practical acidity in gastrointestines of the human broadly ranges from pH 1 to pH 5. Further, even if chitosan is dissolved, the dissolution rate thereof is very sluggish. Upon considering the fact that bisphosphonate is primarily absorbed within 30 minutes after oral administration, a composition of conventional chitosan is not dissolved within this time and thereby exhibits no effects on oral absorption of bisphosphonate drugs. Therefore, the present invention is significantly different from the conventional arts in that chitosan in accordance with the present invention is one that is also rapidly soluble in water unlike conventional chitosan, and in that a pharmaceutical composition in accordance with the present invention is a pharmaceutical composition of bisphosphonate having immediate-release properties using water-soluble chitosan.

European Patent Laid-Open Publication No. 1302201 proposes a composition containing Eudragit E, which is an aminoalkyl methacrylate copolymer, as an oral absorption enhancer and an acidic material. This patent uses Eudragit E which is an acrylate derivative, in order to improve oral absorptivity, whereas soluble chitosan in accordance with present invention has a saccharide structure which is different from that of Eudragit E. In addition, as well recognized in the art, chitosan is a biocompatible substance and is very suitable for use in a pharmaceutical composition of bisphosphonate drugs exhibiting occurrence of severe gastrointestinal disorders in that it can assist in alleviation of gastrointestinal (GI) toxicity (British Journal of Plastic Surgery, 53, 601-606 (2000)).

On the other hand, prior arts attempting to enhance the oral absorption rate via physical control of a release rate of the active substances by using structural properties of the formulation will be illustrated hereinafter.

US Patent Laid-Open Publication No. 2004/0176328 discloses a solid formulation in which a water-soluble active substance is sealed and encapsulated with a material having a lower melting point than the water-soluble active substance, and the encapsulated particles are supported in the continuous solid phase. U.S. Pat. No. 6,207,197 discloses a gastroretentive microsphere comprising a core containing an active ingredient, a controlled release layer composed of a water insoluble polymer and an outer layer composed of a mucoadhesive cationic polymer. The present invention is different than the above patents from the following point of view: regardless of enhancement in the oral absorption rate by interaction between the drug and chitosan, the above-mentioned drug delivery techniques aim at enhancement of bioavailability via control of a drug release rate by preparation of a formulation having the active ingredient sealed in the form of a capsule or microsphere. Further, cited inventions relate to compositions exhibiting sustained-release effects, whereas the present invention provides an immediate-release pharmaceutical composition in order to substantially improve an oral absorption rate of bisphosphonate. Prolonged gastroretention or sustained-release of bisphosphonate drugs may lead to changes in the oral absorption rate, but at the same time, disadvantageously extends a contact time between bisphosphonate and the intestinal mucous membrane, thereby leading to increased toxicity in the esophagi and gastrointestines. Therefore, such type of drug release is not a suitable controlled-release of the drug.

### DISCLOSURE OF INVENTION

### Technical Problem

Therefore, the present invention has been made in view of the above problems, and it is an object of the present invention to enhance an oral absorption rate of a polar active substance by combining a bisphosphonate drug, i.e. alendronate sodium, which exhibits difficulty to pass through lipid biomembranes of the gastrointestinal tracts due to its high polarity, thus resulting in low bioavailability and therefore is conventionally administered at a high dose unit, with water-soluble, high-molecular weight chitosan and/or low-molecular weight chitosan and/or a chitosan monomer, which are soluble in water, in a suitable mixing ratio.

It is another object of the present invention to provide a pharmaceutical composition of a bisphosphonate drug that minimizes adverse side effects of the drug by lowering the dose unit of the active substance which has been administered at a relatively high dose unit via enhancement of an oral absorption rate and at the same time, that minimizes gastrointestinal (GI) toxicity via combination with water-soluble chitosan having biomembrane therapeutic effects.

It is yet another object of the present invention to design a pharmaceutical formulation providing a solution to sluggish dissolution problems occurring upon formulation into a pharmaceutical preparation due to combination with a polymer having a slow dissolution rate, by formulating into the preparation using the above pharmaceutical composition.

### Technical Solution

In accordance with an aspect of the present invention, the above and other objects can be accomplished by the provision of a pharmaceutical composition comprising at least one active substance selected from bisphosphonate drugs which are ionized and highly water-soluble in vivo and therefore do not pass through a lipid biomembrane, and has bioavailability of less than 10%, and at least one selected from biocompatible water-soluble chitosan, as essential ingredients, whereby the composition is rapidly disintegrated and releases more than 80% of the active substance into a gastrointestinal system within at least 60 minutes, thereby leading to an enhanced oral absorption rate of the bisphosphonate drug.

Features of the present invention are broadly classified into three points. Firstly, the present invention facilitates absorption of the drug through an intercellular space by relatively exposing a cationic polar portion of the drug by masking an anionic polar portion of the polar active substance (bisphosphonate drug) with a cationic charge portion of water-soluble chitosan which is also soluble in water. Alternatively, by decreasing polarity of the drug and increasing hydrophobicity thereof using water-soluble chitosan and/or low-molecular weight chitosan and/or a chitosan monomer, bioavailability of the drug is improved via facilitated delivery of the active substance to the inside of the lipid biomembrane by induction of conditions that are advantageous for free diffusion and distribution through the lipid membrane. Secondly, the present invention has attempted to reduce esophageal and gastrointestinal toxicity exhibited by preparations of conventional bisphosphonate drugs, via reduction of a dose unit of the active substance, in conjunction with use of chitosan, particularly water-soluble chitosan, which is known to have wound-healing effects via stimulation of fibroblast proliferation and is known to reduce GI toxicity (Biomaterials, 20, 2959-2966 (2001) & Advanced Drug Delivery Review, 52, 105-115 (2001)). The water-soluble chitosan applied in the present invention has preferably solubility in water of more than 0.1% (w/v) and more preferably more than 0.5% (w/v).

Finally, when a drug is formulated using conventional chitosan which is insoluble in water and is soluble only under acidic conditions, a dissolution rate of the drug is slow or dissolution thereof itself is incomplete due to individual difference and thereby desired absorption effects of the drug are not exerted within 60 minutes and preferably 30 minutes in which prevalent absorption of the drug occurs. Therefore, the present invention has induced fast release of the active substance by preparing an immediate-disintegration preparation which is also rapidly dissolved in the stomach, via use of water-soluble chitosan which is readily soluble in water, instead of using conventional chitosan.

In conclusion, in accordance with the present invention, by increasing an oral absorption rate of bisphosphonate drugs which have difficulty in being orally absorbed, it is possible to develop a novel preparation of the bisphosphonate drug which effectively prevents bone absorption-inhibitory action even with use of a small unit dose of the drug, and reduces esophageal and gastrointestinal toxicity caused by the drug, thereby providing convenient adaptability of a patient.

The composition in accordance with the present invention essentially comprises a bisphosphonate drug and a biocompatible cationic polymer, i.e., water-soluble chitosan and/or low-molecular weight chitosan and/or a chitosan monomer, and may further comprise a pharmaceutically acceptable excipient.

The bisphosphonate drug in accordance with the present invention refers to a drug exhibiting incapability of oral absorption due to high water-solubility thereof, and having bioavailability of less than 30%, particularly less than 10% and having one or more anionic charges and/or cationic charges in a structure of the drug when it is dissolved in water. In addition, the bisphosphonate drug usually has a water partition coefficient (Log P) of not more than 1.0 and thus exhibits a relatively high affinity for water as compared to oil. Examples of such active substances may include alendronate (4-Amino-1-hydroxybutylidene-1,1-biphosphonate), cimadronate (incadronate, 1-(cycloheptylamino)methylidene-1,1-bisphosphonate), etidronate (1-hydroxyethylidene-1,1-bisphosphonate), tiludronate (1-(4-chlorophenylthio)methylidene-1,1-bisphosphonate), minodronate (1-hydroxy-2-(imidazo[1,2-a]pyridin-3-yl)ethylidene-1,1-bisphosphonate), ibandronate (1-hydroxy-3-(N-methyl-N-pentylamino)propylidene-1,1-bisphosphonate), risedronate (1-hydroxy-2-(3-pyridyl)ethylidene-1,1-bisphosphonate), pamidronate (3-amino-1-hydroxypropylidene)-1,1-bisphosphonate), zoledronate (1-hydroxy-3-(1-pyrrolidinyl)propylidene-1,1-bisphosphonate, 1-hydroxy-2-(1-imidazolyl)ethylidene-1,1-bisphosphonate), neridronate (6-amino-1-hydroxyhexilidene-1,1-bisphosphonate), olpadronate (mildronate, N,N-dimethyl-3-amino-1-hydroxypropylidene-1,1-bisphosphonate) and salts or ester derivatives thereof and mixtures thereof.

The bisphosphonate drug is an osteoporosis therapeutic drug that strongly inhibits resorption of calcium from the bone tissue to thereby deteriorate functions of osteoclasts, thus being capable of increasing bone density. However, the bisphosphonate drug has superior efficacy, but is recognized as a drug suffering from many problems associated with in vivo application thereof. For oral absorption, the bisphosphonate drug should be taken in conjunction with excessive amounts of water 30 minutes prior to breakfast in order to achieve drug absorption, and even then bioavailability thereof is less than 30%, particularly less than 10%. The most representative bisphosphonate drug, alendronate is reported to have bioavailability of less than 0.7%. In particular, it is also known that alendronate strongly binds to multivalent metal ions such as calcium, thereby forming precipitates, and bioavailability thereof is decreased to less than a half by intake of meals or beverages. Regarding usage of the drug, when a tablet is dissolved and administered in the mouth, this may cause serious mouth ulcers or hepatitis. Even when it is normally taken, long-term treatment results in incidence of heartburn or epigastric pain in about 36% of patients. In the cases of esophageal diseases, prescription manual states possibility of severe esophagitis or extensive esophagitis.

As such, the most suitable incrementally modified drugs (IMDs) of the bisphosphonate drugs are pharmaceutical compositions which minimize adverse side effects accompanied by the drug itself by decreasing a unit dose of the drug exerting equivalent effects via improvement of bioavailability thereof, and further contain a biocompatible substance that does not increases GI toxicity or can decrease toxicity.

After it is distributed into the bone tissue, bisphosphonate exhibits a very long-term half life of 11.9 years and it is thus recognized that administration at an interval of 5 days or longer during which gastrointestinal mucous tissues are regenerated is an administration period minimizing adverse side effects without decreased efficacy. Therefore, in the case of alendronate, oral administration of 70 mg unit dose at an interval of every 7 days rather than 10 mg every day is a method capable of alleviating side effects of the drug. However, upon considering that this method also suffers from low bioavailability of less than 1%, greatly reducing the unit of 70 mg administered at an interval of every 7 days, by increasing bioavailability of the drug, may be the most efficient method solving the disadvantages and problems associated with bisphosphonate drugs.

As an absorption carrier that is a biocompatible material and is capable of improving bioavailability of bisphosphonate, water-soluble chitosan is suitable. The water-soluble chitosan in accordance with the present invention is a concept covering high-molecular weight chitosan, mixed chitosan according to different molecular weights, low-molecular weight chitosan, a chitooligosaccharide, a chitosan monomer (glucosamine) and mixtures thereof, which are soluble in water, and includes a mixture in which chitosan and an acidic material are homogeneously blended. Water-soluble chitosan utilized in the present invention is also very rapidly dissolved in water without any other derivatization process or an additional acidification process. Glucosamine, a monomer of chitosan, exhibits very high solubility in water of more than 0.5% (w/v) and can impart a constant increase in a body's absorption rate regardless of acidity in the gastrointestines of the living organisms.

Water-soluble chitosan in accordance with the present invention consists of glucosamine monomers, and may be broadly classified into water-soluble, high-molecular weight chitosan, low-molecular weight chitosan and chitosan monomer (glucosamine), depending upon molecular weight. The water-soluble, high-molecular weight chitosan that can be used in the present invention has a molecular weight of 10,000 to 500,000, and preferably 100,000 to 200,000. The water-soluble, low-molecular weight chitosan that can be used in the present invention is used interchangeably with chitooligosaccharide in the equivalent meaning throughout the description of the present invention, and has a molecular weight of 200 to 10,000, and preferably 500 to 2,000. The water-soluble chitosan monomer literally means glucosamine (chitosamine, 2-amino-2-deoxy-D-glucose) which is produced by decomposition of chitosan into a monomer unit thereof.

Such water-soluble chitosan is a mixture in which chitosan is homogeneously blended with an acidic material, or an acid salt of chitosan in which the chitosan monomer glucosamine and an acidic material are arranged by means of ionic attraction. As the acidic material, at least one acid selected from inorganic acids such as hydrochloric acid, sulfuric acid and nitric acid or organic acids such as lactic acid, acetic acid, citric acid, tartaric acid and succinic acid may be used. Hydrochloric acid may be preferably used although the present invention is not limited thereto. In the above description, the mixture in which chitosan is homogeneously blended with the acidic material refers to a form in which conventional chitosan is physically and homogeneously mixed with the inorganic acid or organic acid. In the above description, the acid salt of chitosan in which the chitosan monomer glucosamine is arranged with the acidic material by means of ionic attraction refers to a form in which glucosamine and acidic material are regularly arranged by action of ionic attraction between cationic charge and anionic charge because an amine group of glucosamine has the cationic charge and the inorganic acid or organic acid has the anionic charge. Specific examples of the water-soluble chitosan that can be used in the present invention include, but are not limited to, HFP Chitosan (JAKWANG Co., Ltd., Korea) and FACOS (KITTOLIFE Co., Ltd., Korea).

In addition, the water-soluble chitosan in accordance with the present invention also encompasses a solid or liquid mixture in which the chitosan monomer glucosamine and the inorganic acid or organic acid are homogeneously admixed in a molar ratio of 1:0.1 to 1:50 and preferably 1:0.5 to 1:10, or chitosan powder obtained by dissolving all of the materials in water, followed by drying. The chitosan powder, which was obtained by dissolving all of conventional chitosan and inorganic acid or organic acid in water and then adding an organic solvent to the resulting solution, followed by precipitation and removal of the organic solvent, also falls within the scope of the water-soluble chitosan in accordance with the present invention.

Conventional chitosan, which is obtained by deacetylation of chitin from protective tissues of exoskeletons or cell walls of a crustacean including crabs and shrimps and insects, is also composed of glucosamine as a constituting monomer, but does not exhibit homogeneous distribution of acidic materials or does not contain the acidic materials in the monomer structure thereof, as compared to water-soluble chitosan in accordance with the present invention. As a result, such conventional chitosan is soluble only under acidic conditions of less than pH 3 and is completely insoluble in water, and even in the acidic solution, the dissolution rate thereof is also very slow, thereby failing to achieve an enhanced oral absorption rate which is an effect of the present invention.

Even though gastrointestinal environment is acidic, practical acidity in the human gastrointestine exhibits a broad range of pH 1 to pH 5 and therefore there is high possibility that conventional chitosan is not dissolved and even if it is dissolved, the dissolution rate thereof is very low. Consequently, with use of conventional chitosan, it is impossible to achieve substantial improvement of the oral absorption rate in bisphosphonate drugs, rapid absorption of which is suitably within 1 hour upon in vivo application. Further, conventional chitosan having a slow dissolution rate inhibits release of the drug and thereby may also cause adverse effects that result in decreased absorption of bisphosphonate drugs requiring rapid absorption.

In general, in order to prepare a pharmaceutical composition in accordance with the present invention, the active substance may be mixed with water-soluble chitosan and optionally a pharmaceutically acceptable excipient. The active substance is admixed with at least one selected from water-soluble, high-molecular weight chitosan and/or low-molecular weight chitosan (chitooligosaccharide) and/or a chitosan monomer in a weight ratio of 100:1 to 1:100, preferably 25:1 to 1:25 and more preferably 10:1 to 1:10, based on the weight of the active substance. In addition, the pharmaceutical composition in accordance with the present invention can be formulated by pharmaceutically acceptable conventional methods such as blending and kneading, grinding, sieving, filling, compressing, lyophilization, spray-drying, fluid-bed drying, centrifugal granulation or the like.

The pharmaceutical composition in accordance with the present invention may further contain pharmaceutically acceptable excipients which are commonly used in the art. As the pharmaceutically acceptable excipients, diluent, binder, disintegrant, coloring agent, sweetening agent, flavor, preservative, lubricant and the like may be used. In addition, excipients having mixed-functions may also be employed. Specific examples of the pharmaceutically acceptable excipients that can be utilized in the present invention include, but are not limited to, the followings: the diluent may be selected from lactose, dextrose, microcrystalline cellulose, starch and any combination thereof; the binder may be selected from polyvinylpyrrolidone, hydroxypropyl cellulose, hydroxypropylmethyl cellulose, hydroxyethyl cellulose, dicalcium phosphate, sodium alginate and any combination thereof; the disintegrant may be selected from sodium croscamelose, sodium starch glycolate, cross-linked polyvinylpyrrolidone, pregelatinized starch, lower substituted hydroxypropyl cellulose and any combination thereof; the coloring agent may be selected from water-soluble pigment, tar dyes and any combination thereof; the sweetening agent may be selected from dextrose, sorbitol, mannitol, aspartame, acesulfam, citric acid and any combination thereof; the flavor may be selected from orange flavor powder, grape flavor powder, strawberry flavor powder, blueberry flavor powder and any combination thereof; the preservative may be selected from benzoic acid, methylparaben, ethylparaben, propylparaben and any combination thereof; and the lubricant may be selected from magnesium stearate, talc, light anhydrous silicic acid, sucrose fatty acid ester and any combination thereof. The composition of the thus-formed bisphosphonate drug and water-soluble chitosan is rapidly disintegrated in the stomach, thereby completely releasing the bisphosphonate drug from the pharmaceutical formulation within one hour, preferably within 30 minutes, and chitosan is rapidly dissolved, thereby facilitating permeation of the drug to the biomembrane.

The composition in accordance with the present invention may be designed into solid formulations such as powder, granules, tablets and capsules by homogeneously mixing it in a solid phase in the absence of water at the same ratio as described above. The composition in accordance with the present invention may be designed into liquid formulations such as syrup and solution by dissolving the composition in conjunction with water. If necessary, the composition may lyophilized and then suspended, emulsified or dissolved for reconstitution prior to use.

The pharmaceutical composition in accordance with the present invention exhibits a remarkably increased oral absorption rate as compared to conventional compositions and therefore can be used at significantly reduced unit dose. For example, where the active ingredient is alendronate sodium, dose thereof can be reduced to a maximum of a hundredth of conventional unit dose.

### DESCRIPTION OF THE DRAWINGS

The above and other objects, features and other advantages of the present invention will be more clearly understood from the following detailed description taken in conjunction with the accompanying drawings, in which:

FIG. 1 is a graph showing permeation ratios (%) of active drugs to Caco-2 cells, measured in Experimental Examples 1 through 7; and

FIG. 2 is a graph showing the results of dissolution ratio (%) of tablets which were prepared with compositions in accordance with the present invention in Experimental Examples 8 through 12, measured with respect to the passage of time.

### BEST MODE

### EXAMPLES

Now, the present invention will be described in more detail with reference to the following examples. These examples are provided only for illustrating the present invention and should not be construed as limiting the scope and sprit of the present invention.

### Examples 1 through 3

For Examples 1 through 3, 13.0 mg of alendronate sodium, as an active substance (drug), was dissolved in 100 ml of HBSS buffer, and 6.5 mg, 26.0 mg, 52.0 mg of water-soluble, high-molecular weight chitosan (HFP®, JAKWANG Co., Ltd., Korea) were dissolved in 100 ml of HBSS buffer, respectively. Thereafter, equal aliquots were taken from the alendronate solution and the respective chitosan solutions and were homogeneously mixed with stirring.

### Example 4

For this example, 13.0 mg of alendronate sodium, as an active substance, was dissolved in 100 ml of HBSS buffer, and 52.0 mg of water-soluble, low-molecular weight chitosan (FACOS®, KITTOLIFE Co., Ltd., Korea) was dissolved in 100 ml of HBSS buffer. Thereafter, equal aliquots were taken from two solutions and were homogeneously mixed with stirring.

### Example 5

For this example, 13.0 mg of alendronate sodium, as an active substance, was dissolved in 100 ml of HBSS buffer, and 34.5 mg of glucosamine hydrochloride, which is a chitosan monomer, was dissolved in 100 ml of HBSS buffer. Thereafter, equal aliquots were taken from two solutions and were homogeneously mixed with stirring.

### Comparative Examples 1 and 2

For Comparative Example 1, 13.0 mg of alendronate sodium, as an active substance, was dissolved in 200 ml of HBSS buffer.

For Comparative Example 2, 13.0 mg of alendronate sodium, as an active substance, was dissolved in 100 mml of HBSS buffer, and 52.0 mg of conventional high-molecular weight chitosan was suspended in 100 ml of HBSS buffer.

All of the solutions prepared in Examples 1 through 5 and Comparative Example 1 were dissolved so they were transparent to naked eyes, but the solution prepared in Comparative Example 2 was not completely dissolved to naked eyes and used in opaque suspension state for Experimental Examples which will be illustrated hereinafter.

### Experimental Examples 1 through 7

For Experimental Examples 1 through 7, each 200 μl aliquot of the solutions and suspension, which were prepared in Examples 1 through 5 and Comparative Examples 1 and 2, was added to an apical side of 2-week cultured Caco-2 cells, and 1,000 μl of HEPES buffer was placed in basolateral side of Caco-2 cells, and cells were then cultured in an incubator for 2 hours. After 2 hours, buffer was recovered from both apical and basolateral sides of cells and samples were pre-treated with FMOC-Cl. Then, a concentration of the drug was determined by high-speed liquid chromatography, and a degree of absorption from the apical side to the basolateral side was determined according to the following equation:

$$Permeation ratio ( \% ) = \frac{Permeated amount of basolateral side ( ng )}{Loaded amount of apical side ( ng )}$$

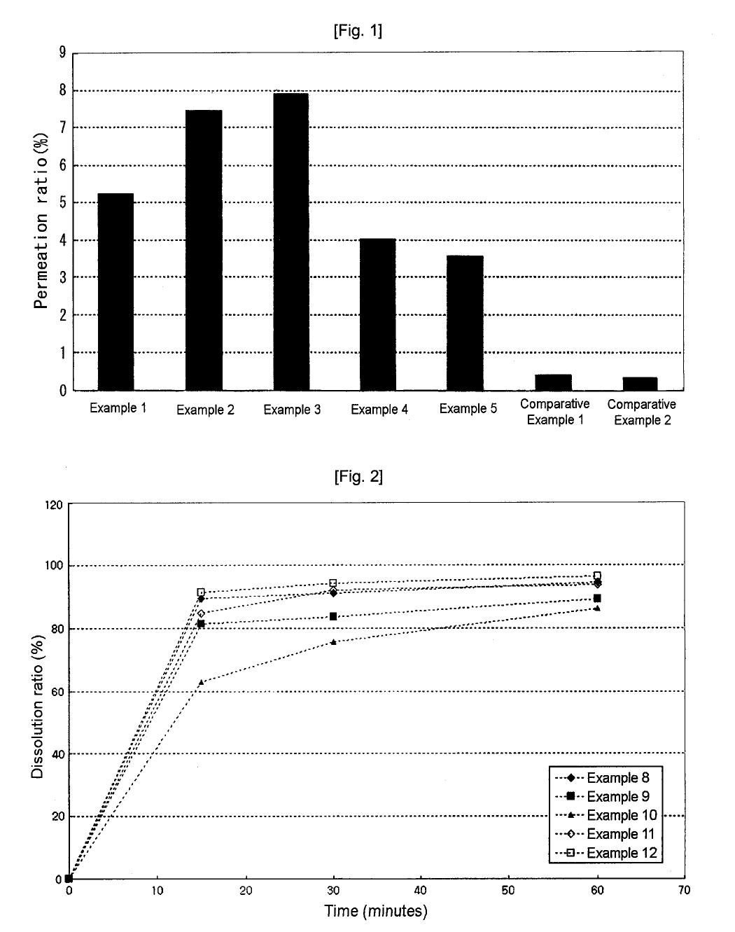

Permeation experiment with Caco-2 cells is a typical experimental model which is carried out so as to evaluate an oral absorption rate of the drug and an absorption mechanism via an oral route in the art. FIG. 1 is a graph showing % absorption of bisphosphonate 2 hours after Caco-2 cell experiments of the composition of the present invention were carried out in Experimental Examples 1 through 7. Herein, Experimental Examples 1 through 5 exhibited a remarkable 8 to 20-fold increase in absorption rate, as compared to Experimental Example 6 using the solution of Comparative Example 1. That is, it can be seen that the composition in accordance with the present invention has resulted in a significantly increased oral absorption rate for an active substance having an oral absorption rate of less than about 1% due to high polarity thereof. Whereas, Experimental Example 7 using the suspension of Comparative Example 2 exhibited a permeation ratio of the drug similar to that of Comparative Example 1 and thereby it was believed that conventional chitosan is insoluble in water and thereby has no effect on absorption of the drug. Meanwhile, it can be seen that the water-soluble, high-molecular weight chitosan (HFP), low-molecular weight chitosan and a chitosan monomer, which were developed to have easy solubility and high bioavailability, have effects on cell permeability of alendronate sodium, thereby resulting in a remarkable increase of the absorption rate of the drug upon oral administration.

### Examples 6 through 8

For Examples 6 through 8, 5.02 g of alendronate sodium as an active substance was mixed with 2.51 g, 7.53 g and 20.08 g of water-soluble, high-molecular weight chitosan (HFP®), respectively, and as a conventional disintegrant among pharmaceutically acceptable excipients, 5% (w/w) sodium starch glycolate was added thereto, followed by homogeneous mixing. The resulting mixtures were compressed and crushed to powder, and were then screened through an 18-mesh sieve, thereby forming granules. 5% (w/w) sodium starch glycolate as the disintegrant and 3% (w/w) glyceryl behenate as the lubricant were added to respective granules and a proper quantity of microcrystalline cellulose was added and finally mixed to the resulting mixtures, such that the total weight is 70 g. The thus-formed final mixtures were compressed to contain 20 mg of alendronic acid as an active ingredient, using a tableting machine, thereby preparing respective tablets.

### Example 9

For Example 9, 5.02 g of alendronate sodium as an active substance was mixed with 20.08 g of water-soluble, low-molecular weight chitosan (FACOS®), and as a conventional disintegrant among pharmaceutically acceptable excipients, 5% (w/w) sodium starch glycolate was added thereto, followed by homogeneous mixing. The resulting mixture was kneaded with absolute ethanol and dried, and was then screened through an 18-mesh sieve, thereby forming granules. 5% (w/w) sodium starch glycolate as the disintegrant and 3% (w/w) glyceryl behenate as the lubricant were added to the thus-formed granules and a proper quantity of microcrystalline cellulose was added and finally mixed to the resulting mixture, such that the total weight is 70 g. The thus-formed final mixture was compressed to contain 20 mg of alendronic acid as an active ingredient, using a tableting machine, thereby preparing respective tablets.

### Example 10

For Example 10, 5.02 g of alendronate sodium as an active substance was mixed with 13.3 g of the chitosan monomer glucosamine hydrochloride, and as a conventional disintegrant among pharmaceutically acceptable excipients, 5% (w/w) sodium starch glycolate was added thereto, followed by homogeneous mixing. The resulting mixture was kneaded with water and dried, and was then screened through an 18-mesh sieve, thereby forming granules. 5% (w/w) sodium starch glycolate as the disintegrant and 3% (w/w) glyceryl behenate as the lubricant were added to the thus-formed granules and a proper quantity of microcrystalline cellulose was added and mixed to the resulting mixture, such that the total weight is 70 g. The thus-formed final mixture was compressed to contain 20 mg of alendronic acid as an active ingredient, using a tableting machine, thereby preparing respective tablets.

### Examples 11 Through 13

For Examples 11 through 13, 5.02 g of alendronate sodium as an active substance was mixed with 2.51 g, 7.53 g and 20.08 g of water-soluble, high-molecular weight chitosan, respectively, and granules were then prepared in the same manner as Examples 6 through 8, followed by final mixing. The resulting final mixture was filled to contain 20 mg of alendronic acid as an active ingredient, using a capsule filler, thereby preparing respective capsules.

### Examples 14

For Example 14, 5.02 g of alendronate sodium as an active substance was mixed with 20.08 g of water-soluble, low-molecular weight chitosan, and granules were then prepared in the same manner as Example 9, followed by final mixing. The resulting final mixture was filled to contain 20 mg of alendronic acid as an active ingredient, using capsule filler, thereby preparing capsules.

### Examples 15

For Example 15, 5.02 g of alendronate sodium as an active substance was mixed with 13.3 g of the chitosan monomer glucosamine hydrochloride, and granules were then prepared in the same manner as Example 10, followed by final mixing. The resulting final mixture was filled to contain 20 mg of alendronic acid as an active ingredient, using capsule filler, thereby preparing respective capsules.

### Example 16

For Example 16, 1.0 g of alendronate sodium as an active substance and 4.0 g of water-soluble, high-molecular weight chitosan (HFP®) were added to 200 ml of water and the mixture was dissolved with stirring until it was transparent to naked eyes. Then, 50 mg of acesulfam and 20 mg of citric acid, and orange essence (q.s.) were added to the thus-prepared solution, thereby preparing a liquid oral preparation.

### Example 17

For Example 17, 1.0 g of alendronate sodium as an active substance and 4.0 g of water-soluble, low-molecular weight chitosan (FACOS®) were added to 200 ml of water and the mixture was stirred until it was transparent to naked eyes. Then, 50 mg of acesulfam, 20 mg of citric acid and orange essence (q.s.) were added to the thus-prepared solution, thereby preparing a liquid oral preparation.

### Examples 18 and 19

For Examples 18 and 19, 5.02 g of alendronate sodium as an active substance and 20.08 g of chitosan were mixed with 10.04 g of glutamic acid and 10.04 g of citric acid, as organic acids, respectively, and as a conventional disintegrant among pharmaceutically acceptable excipients, 5% (w/w) sodium starch glycolate was added thereto, followed by homogeneous mixing. The resulting mixtures were kneaded with absolute ethanol and dried, compressed and crushed into powder, and were then screened through an 18-mesh sieve, thereby forming granules. 5% (w/w) sodium starch glycolate as the disintegrant and 3% (w/w) glyceryl behenate as the lubricant were added to respective granules, and a proper quantity of microcrystalline cellulose was added and finally mixed to the resulting mixtures, such that the total weight is 70 g. The thus-formed final granules were compressed to contain 20 mg of alendronic acid as an active ingredient, using a tableting machine, thereby preparing respective tablets.

### Experimental Examples 8 through 12

For Experimental Examples 8 through 12, using a dissolution machine operating at 37° C. and 50 rpm, and using tablets which were prepared in Examples 6 through 10, dissolution ratio of the respective tablets in water were determined according to the following equation:

$$Dissolution ratio ( \% ) = \frac{{A}_{t}}{{A}_{s}} \times \frac{Standard collected ( mg )}{91.37} \times 9 \times Purity of Standard ( \% )$$

Aₜ: Peak area of test liquid per dissolution time

Aₛ: Peak area of standard liquid per dissolution time

FIG. 2 is a graph showing the results of dissolution ratio (%) of tablets which were prepared with compositions in accordance with the present invention in Experimental Examples 8 through 12, measured with respect to time. Here, all of Experimental Examples 8 through 12 exhibited a high dissolution ratio of more than 80% within 60 minutes after initiation of dissolution. That is, when they were orally administered in conjunction with water, preparations of the present invention showed that most of drugs were released from the preparations within 1 hour, and therefore it could be seen that the present invention will contribute to improvement in an absorption rate of drugs unlike conventional chitosan that retards release of the drug due to poor solubility thereof in water.

### INDUSTRIAL APPLICABILITY

The present invention is directed to a pharmaceutical composition comprising at least one active substance selected from bisphosphonate drugs which are ionized and highly water-soluble in vivo and therefore do not pass through a lipid biomembrane, and has bioavailability of less than 10%, and at least one selected from biocompatible water-soluble chitosan, as essential ingredients. The present invention improves oral absorption of bisphosphonate drugs which have been administered at high doses, thereby minimizing adverse side effects of drugs and therefore being capable of enhancing adaptability of patients to the drug of interest.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A pharmaceutical composition having an improved oral absorption rate, comprising at least one active substance selected from a bisphosphonate drug, and at least one selected from water-soluble chitosan having a solubility in water of more than 0.1% (w/v), in a weight ratio of 25:1 to 1:25.

2. The composition according to claim 1, wherein the bisphosphonate drug and the water-soluble chitosan are mixed in a weight ratio of 10:1 to 1:10.

3. The composition according to claim 1, wherein the water-soluble chitosan has a solubility in water of more than 0.5% (w/v).

4. The composition according to claim 1, wherein the water-soluble chitosan is at least one selected from high-molecular weight chitosan having a molecular weight of 10,000 to 500,000, low-molecular weight chitosan having a molecular weight of 200 to 10,000, glucosamine and mixtures thereof.

5. The composition according to claim 1, wherein the water-soluble chitosan is a homogeneous mixture of chitosan and an acidic material.

6. The composition according to claim 1, wherein the water-soluble chitosan is an acid salt of chitosan in which the chitosan monomer glucosamine and an acidic material are arranged by ionic attraction.

7. The composition according to claim 1, wherein the bisphosphonate drug is at least one selected from alendronate, cimadronate (incadronate), etidronate, tiludronate, ibandronate, risedronate, pamidronate, zoledronate, neridronate, olpadronate (mildronate), minodronate and salts or ester derivatives thereof.

8. The composition according to claim 1, wherein the bisphosphonate drug is alendronate sodium.

9. The composition according to claim 1, wherein the composition is formulated as powder, granule, tablet, capsule, syrup, solution, or lyophilizate that is suspended, emulsified or dissolved for use prior to administration.
